(19) United States
(12) Patent Application Publication
(10) Pub. No.: US 2024/0126097 A1
(43) Pub. Date: Apr. 18, 2024

(54) GLASSES

(71) Applicant: UVEX ARBEITSSCHUTZ GMBH, Fürth (DE)

(72) Inventors: Martin WOLF, Ansbach (DE); Florian KÜHNLEIN, Veitsbronn (DE); Michael ABEL, Feucht (DE); Frederic SCHUSS, Nürnberg (DE)

(21) Appl. No.: 18/486,658

(22) Filed: Oct. 13, 2023

(30) Foreign Application Priority Data
Oct. 13, 2022 (DE) 10 2022 210 819.1

(51) Int. Cl. G02C 5/00; G02C 5/12

(52) U.S. Cl. CPC G02C 5/006; G02C 5/12

(57) ABSTRACT

Glasses have a lens arrangement, a glasses frame, and at least one pivot point for joining together the lens arrangement and the glasses frame by pivoting them relative to one another, wherein the glasses frame has a nose support device for support against a nose of a glasses wearer, and wherein the lens arrangement has a nose support device receptacle which, in the joined-together state of the lens arrangement and the glasses frame, receives the nose support device.

### CROSS-REFERENCES TO RELATED APPLICATIONS

This application claims the priority of German Patent Application, Serial No. 10 2022 210 819.1, filed 13 Oct. 2022, the content of which is incorporated herein by reference in its entirety as if fully set forth herein.

### FIELD OF THE INVENTION

The invention relates to glasses, in particular safety glasses. They can also be designed as sports glasses, optical glasses or the like, for example. The invention is furthermore directed to a method for joining together or assembling glasses of this kind.

### BACKGROUND OF THE INVENTION

Glasses with interchangeable individual parts are widely known from the prior art. Special aids or tools are often required to join the glasses together. Moreover, the individual parts generally have complex geometries.

US 2005/0036104 A1, U.S. Pat. No. 5,815,235, US 2013/0185849 A1 and US 2006/0279692 A1 disclose glasses of the prior art.

### SUMMARY OF THE INVENTION

The invention is based on an object of providing glasses which, on the one hand, are relatively simple to produce and, on the other hand, are extremely easy to assemble.

According to the invention, this object is achieved by glasses having a lens arrangement, having a glasses frame, and having at least one pivot point for joining together the lens arrangement and the glasses frame by pivoting them relative to one another, wherein the glasses frame has a nose support device for support against a nose of a glasses wearer, wherein the lens arrangement has a nose support device receptacle which, in a joined-together state of the lens arrangement and the glasses frame, receives the nose support device.

According to the invention, this object is further achieved by a method for joining together the glasses according to the invention, comprising the step of pivoting the lens arrangement and the glasses frame relative to one another about at least one pivot point.

At the heart of the invention is the joining together or assembly of the lens arrangement and the glasses frame by pivoting them relative to one another about or by way of at least one, preferably precisely one, pivot point. For example, the at least one pivot point provides at least one, preferably precisely one, pivot axis, or at least one, preferably precisely one, pivoting range for the, in particular at least partially guided, joining together of the lens arrangement and the glasses frame.

To join together the lens arrangement and the glasses frame, the lens arrangement is pivoted relative to the glasses frame, or the glasses frame is pivoted relative to the lens arrangement, for example. As another example, the lens arrangement and the glasses frame are pivoted jointly in an appropriate manner relative to one another.

It is expedient if the joining together of the lens arrangement and the glasses frame is possible or can be carried out manually and, expediently, without tools, i.e. without aids. It can preferably be carried out intuitively. To achieve this, it is preferably possible to avoid touching the lens arrangement in the viewing region thereof. It is advantageous if separate connecting means are no longer necessary.

The lens arrangement comprises precisely one integral, continuous lens, for example. As an alternative, this has two lenses, for example. The lens arrangement is preferably exchangeable.

The glasses frame is preferably capable of holding or carrying the lens arrangement when the lens arrangement and the glasses frame have been joined together. It then expediently runs at least partially around the lens arrangement and preferably has a lens arrangement receptacle for receiving the lens arrangement which is at least in part spatially delimited towards the outside. It is advantageous if the lens arrangement receptacle is open towards the bottom. It is expedient if the glasses frame is formed from plastic. It is preferably an injection moulding.

It is expedient if the glasses have a head-holding device for holding the lens arrangement on a glasses wearer. The head-holding device is preferably arranged on the glasses frame. It is formed, for example, by at least one holding strap, which runs around the back of the head of the glasses wearer when the glasses are being worn. Alternatively, it is formed by two temples, in particular pivotable temples, for example.

The lens arrangement and the glasses frame can preferably be separated from one another again. It is expedient if the separation of the lens arrangement and the glasses frame from one another or the disassembly thereof takes place (substantially) analogously to the joining together of the lens arrangement and the glasses frame, preferably without tools.

It is advantageous if the glasses are symmetrical with respect to a main plane.

A nose support device for supporting the glasses frame against a nose of the glasses wearer leads to particularly high wearing comfort. It is expedient if it is flexible, at least in some region or regions. The nose support device is preferably an integral part of the glasses frame.

The nose support device receptacle and the nose support device are preferably designed to be complementary to one another. The nose support device is preferably (substantially) V-shaped or Y-shaped.

The refinement, in which the at least one pivot point is predefined, allows controlled or defined pivoting of the lens arrangement and of the glasses frame relative to one another to join them together. In this way, they can be joined together in a way which is particularly functionally reliable and easily controllable, and which is user-friendly.

The lens arrangement having at least one lower pre-centring pivoting means, and the glasses frame having at least one lower counterpart pre-centring pivoting means for interacting with the at least one lower pre-centring pivoting means upon joining together the lens arrangement and the glasses frame, ensure the predetermined or defined pivoting of the lens arrangement and the glasses frame relative to one another for joining them together. They are preferably designed to be complementary to one another, at least in an interaction region. The at least one lower pre-centring pivoting means is preferably arranged in a lateral edge or end region of the lens arrangement. It is expedient if a lower pair of pre-centring pivoting means and a lower pair of counterpart pre-centring pivoting means are present.

The at least one lower pre-centring pivoting means is designed as a receptacle, in particular a keyhole-like receptacle, in particular a keyhole-like latching receptacle. The receptacle is preferably open towards the bottom. It is expediently delimited at the top and laterally.

The at least one lower counterpart pre-centring pivoting means is preferably designed as a body and advantageously has a cylindrical or partially cylindrical cross section or bearing region. The body is designed as a pin, lug or the like, for example.

The at least one lower pre-centring pivoting means and the at least one lower counterpart pre-centring pivoting means preferably form a first pre-centring pivoting means and counterpart pre-centring pivoting means, respectively. It is expedient if the lower counterpart pre-centring pivoting means is in latching connection, in particular releasably, with the at least one lower pre-centring pivoting means upon joining together the lens arrangement and the glasses frame and in their joined-together state, and this makes assembly particularly easy and leads to particularly functionally reliable glasses. It is advantageous if the at least one lower pre-centring pivoting means is an integral part of the lens arrangement. The at least one lower counterpart pre-centring pivoting means is advantageously an integral part of the glasses frame.

The lens arrangement having at least one upper pre-centring means, and the glasses frame having at least one upper counterpart pre-centring means for interacting with the at least one upper pre-centring means upon joining together the lens arrangement and the glasses frame, provide a stress-resistant, easily achievable connection between the lens arrangement and the glasses frame. At least in an interaction region, for example, they are preferably designed to be complementary to one another. The at least one upper pre-centring means is preferably arranged in a lateral edge or end region of the lens arrangement. It is expedient if an upper pair of pre-centring means and an upper pair of counterpart pre-centring means are present.

The at least one upper pre-centring means is designed as a protrusion. The at least one upper pre-centring means is designed as a web, in particular an abutment web, plate, pin or the like, for example.

The at least one upper counterpart pre-centring means is preferably embodied as a receptacle, abutment surface, abutment flank or the like.

The at least one upper pre-centring means and the at least one upper counterpart pre-centring means preferably form a second pre-centring means and counterpart pre-centring means, respectively. The at least one upper pre-centring means and/or counterpart pre-centring means is/are preferably active or effective after the at least one lower pre-centring pivoting means and/or counterpart pre-centring pivoting means as the lens arrangement and the glasses frame are joined together.

It is advantageous if the at least one upper pre-centring means is an integral part of the lens arrangement. The at least one upper counterpart pre-centring means is advantageously an integral part of the glasses frame.

The refinement, in which the lens arrangement and the glasses frame are connected to one another by positive engagement in at least one upper glasses region in the joined-together state, also leads to glasses which, in the joined-together state of the lens arrangement and of the glasses frame, are particularly stable and stress-resistant. It is expedient if the lens arrangement and the glasses frame are connected directly to one another in the joined-together state. The glasses frame then preferably engages around the lens arrangement, in particular in some region or regions, in particular from above. It is advantageous if a tongue-and-groove connection is then present at least in some region or regions between the lens arrangement and the glasses frame.

The refinement, in which the nose support device and the nose support device receptacle are connected to one another in a bolt-like manner in the joined-together state of the lens arrangement and the glasses frame, also leads to glasses which, in the joined-together state of the lens arrangement and of the glasses frame, are particularly stable and stress-resistant. There is then preferably a positive connection between the nose support device and the nose support device receptacle. For example, the nose support device rests against or is supported at the bottom, top, and/or laterally against the inside of the lens arrangement and the nose support device receptacle.

The design, in which the nose support device is pivotable as a whole for joining together of the lens arrangement and the glasses frame, likewise results in glasses which are particularly stable and stress-resistant. It is expedient if, after pivoting from its starting position or wearing position into an assembly position, the nose support device pivots back or springs back into the starting position, in particular independently.

The nose support device can be pivoted forwards and upwards, for example.

At least in some region or regions, the frame part carrying the nose support device can be twisted or rotated upon itself, at least partially, preferably about its longitudinal centre line, in order to pivot the nose support device. It is preferably elastic there, at least in some region or regions.

The terms “top”, “bottom”, “front”, “rear”, “on the rear side”, “laterally” or the like used herein refer, in particular, to an orientation of the glasses when worn properly by a glasses wearer.

One preferred embodiment of the invention is described below by way of example with reference to the attached drawing.

### BRIEF DESCRIPTION OF THE DRAWING

FIG. 1 shows a perspective view of glasses according to the invention in their fully joined-together state or wearing position,

FIG. 2 shows a section through the glasses illustrated in FIG. 1, which passes through the nose region thereof,

FIG. 3 shows a section through the glasses shown in FIG. 1, which is laterally offset from the section according to FIG. 2,

FIG. 4 shows an exploded view of the glasses shown in FIG. 1,

FIG. 5 shows a rear view of the glasses frame of the glasses shown in FIG. 1, and

FIGS. 6 to 8 show views which illustrate the joining together of the glasses frame and the lens arrangement

### DESCRIPTION OF THE PREFERRED EMBODIMENT

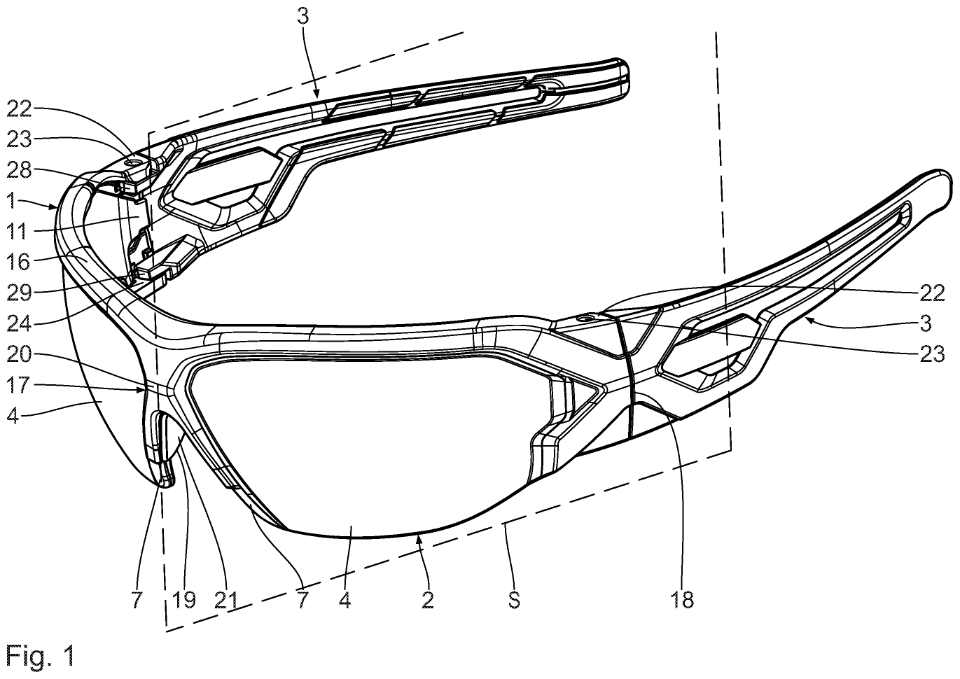

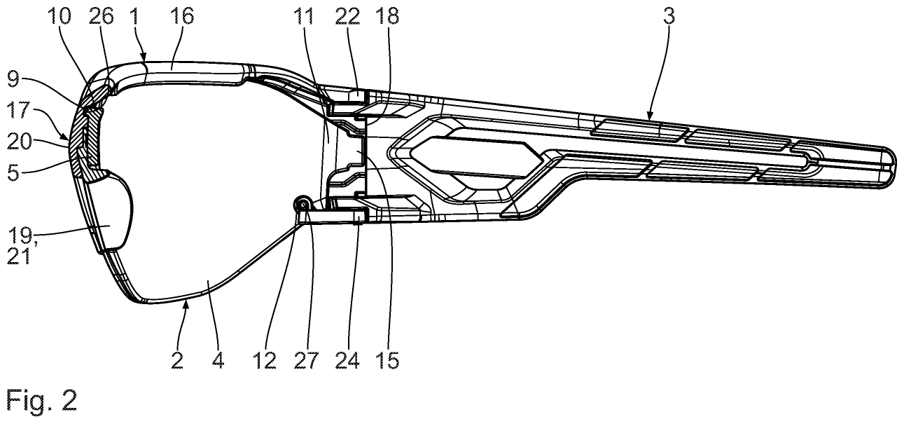

A pair of glasses, which is illustrated in its entirety, in the fully joined-together state, in FIG. 1, comprises a glasses frame 1, a lens arrangement 2 held by the glasses frame 1, and two temples 3, which are pivotally attached at the ends to the glasses frame 1. The glasses are symmetrical with respect to a plane of symmetry S, which passes through the nose region thereof.

The lens arrangement 2 is of one-piece and symmetrical design. It comprises two lenses 4, which are arranged next to one another, and a connecting web 5, which runs between the lenses 4 and connects them to one another in the manner of an arch at the top. The lens arrangement 2 is curved.

The lens arrangement 2 furthermore has two connecting arms 6, which are connected at the bottom to the connecting web 5. The connecting arms 6 extend at a distance from one another and extend downwards from the connecting web 5. They extend away from one another, starting from the connecting web 5. Each connecting arm 6 extends next to a lens 4.

At the bottom, each connecting arm 6 carries a retaining lug or retaining projection or retaining shoulder 7, which projects in the direction of the plane of symmetry S and forms a retaining surface 8 which faces upwards or towards the connecting web 5.

The connecting web 5, the connecting arms 6 and the retaining lugs 7 form or delimit a nose support device receptacle.

At the top on the front, the connecting web 5 carries a latching body 9, which projects forwards and is elongate. It extends perpendicularly to the plane of symmetry S and is, for example, cuboidal or web-shaped.

The lens arrangement 2 further comprises two terminating elements 10 at the top. Each terminating element 10 is, for example, of web-type design and adjoins a lens 4 at the top. Each terminating element 10 extends laterally outwards from the connecting web 5.

In addition, the lens arrangement 2 has two side elements 11, which adjoin the respective terminating element 10 and the respective lens 4 laterally on the outside.

A keyhole-like receptacle 12, which is continuous and open at the bottom, is formed at the bottom in each side element 11. Each receptacle 12 is delimited by two mutually opposite insertion flanks 13, which taper from the bottom upwards towards one another. Each receptacle 12 furthermore has an inner pivot bearing region 14, which adjoins the two insertion flanks 13 and is part-circular or circular in cross section. Each pivot bearing region 14 has a maximum transverse extent which is (slightly) greater than the adjacent (minimum) distance between the insertion flanks 13. Each receptacle 12 forms a first or lower pre-centring pivot receptacle.

Each side element 11 furthermore has a pre-centring means 15, which is arranged above the adjacent receptacle 12. Each pre-centring means 15 is arranged at the edge of the respective side element 11 and projects rearwards. Each pre-centring means 15 forms a second or upper pre-centring means. Each pre-centring means 15 is designed as a protrusion and is, for example, web-like, plate-like, pin-like or similar.

The glasses frame 1 is integral and symmetrical. It extends in a curved manner.

The glasses frame 1 comprises an upper, elongate frame part 16 and a nose support device 17 projecting downwards centrally from the upper frame part 16. Furthermore, the glasses frame 1 has two lateral end elements 18, which adjoin the respective frame part 16 laterally on the outside.

The frame part 16 is designed to be elastic or flexible at least in some region or regions. In particular, when an external force is applied, it can be twisted in a circumferential direction U about its longitudinal centre line, thus enabling the nose support device 17 to be pivoted upwards out of its wearing position (FIG. 1) relative to the end elements 18. The frame part 16 has an angular and/or round cross section, for example.

The nose support device 17 comprises two nose pads 19, which are arranged adjacent to one another, and a connecting body 20 connecting these pads to one another. The nose pads 19 are elongate and extend downwards from the connecting body 20. They extend away from one another, starting from the connecting body 20. Each nose pad 19 has a nose pad surface 21 on the inside, which rests on the nose of a glasses wearer (not shown) when the glasses are worn.

Each end element 18 carries an upper articulation lug 22 with an upper cylindrical bearing recess 23 and a lower articulation lug 24 with a lower cylindrical bearing recess 25. The bearing recesses 23, 25 of each articulation lug 22, 24 are arranged one above the other and provide an articulation axis for the respective temple 3.

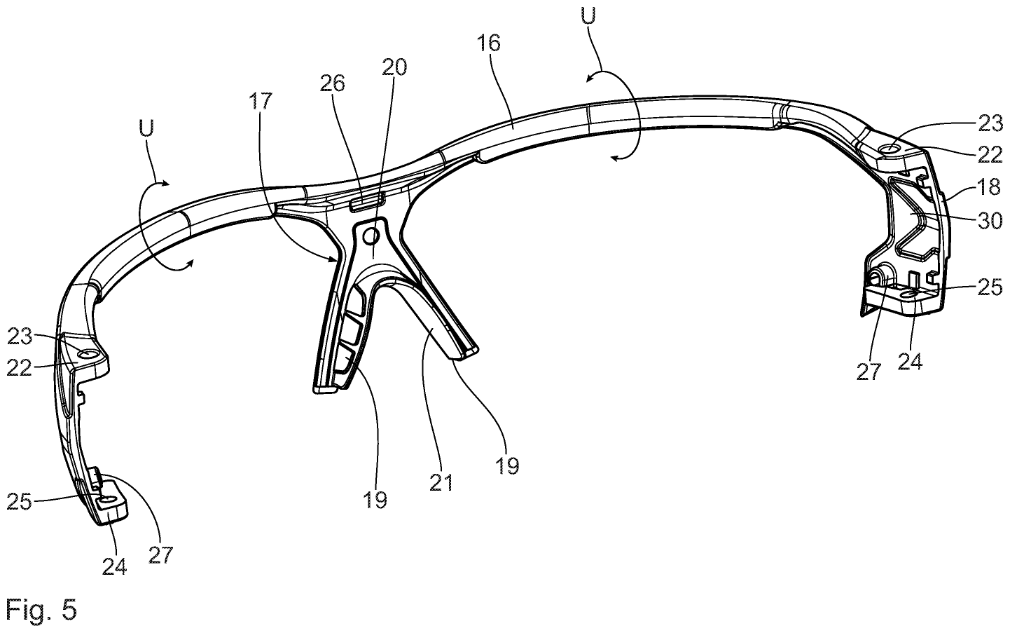

Each end element 18 furthermore carries a pre-centring pin 27 (FIG. 5), which is part-circular or circular in cross section. Each pre-centring pin 27 is arranged above the adjacent lower articulation lug 24. Each pre-centring pin 27 directly adjoins the top of the adjacent lower articulation lug 24, for example. The pre-centring pins 27 are arranged on the inside of the end elements 18. They form lower or first pre-centring pins 27.

In addition, each end element 18 has a pre-centring abutment means 30, which is formed above the adjacent pre-centring pin 27. Each pre-centring abutment means 30 is arranged on the inside of the respective end element 18 and, for example, at the end or at a distance from an outer end of the respective end element 18. The pre-centring abutment means 30 are formed by an inner side or inner flank of the respective end element 18. They are open or delimited at the bottom, top, inside and/or outside, for example. They form upper or second pre-centring abutment means 30.

In addition, the glasses frame 1 has an elongate latching recess 26 (FIG. 5) at the centre of the rear side, which is designed to be complementary to the latching body 9.

Each temple 3 has an upper bearing body 28 and a lower bearing body 29. The bearing bodies 28, 29 are each peg- or pin-shaped. They are arranged one above the other on each temple 3 and, for example, project away from one another. The upper bearing bodies 28 are complementary to the upper bearing recesses 23, while the lower bearing bodies 29 are designed to be complementary to the lower bearing recesses 25. A different arrangement of the bearing bodies 28, 29 and bearing recesses 23, 25 is possible as an alternative. Each temple 3 has only precisely one bearing body 28 or 29, for example.

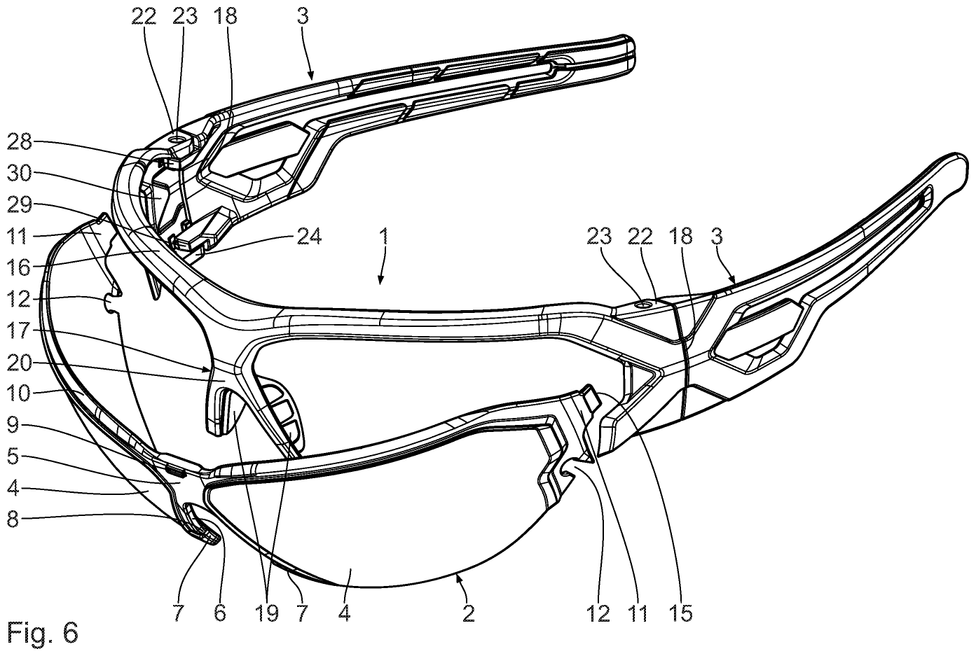

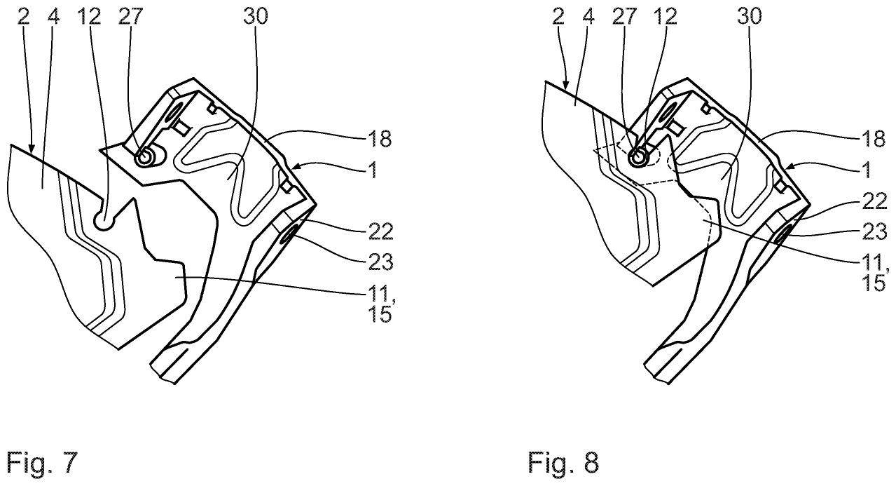

In the following, the joining together of the glasses frame 1, the lens arrangement 2, and the temples 3, i.e. their assembly, will also be described with reference to FIGS. 6 to 8.

The glasses frame 1, the lens arrangement 2, and the temples 3 are originally separate.

First of all, the pre-centring pins 27 are guided into the pivot bearing regions 14 from below via corresponding insertion openings of the receptacles 12 and the insertion flanks 13, preferably simultaneously or uniformly. Alternatively or additionally, the receptacles 12 are moved in corresponding fashion relative to the pre-centring pins 27.

During this process, the pre-centring pins 27 temporarily press the insertion flanks 13 away from one another and latch or snap into the respective pivot bearing region 14. Alternatively or additionally, the pre-centring pins 27 can be pressed together in order to enter the respective pivot bearing region 14. Each pivot bearing region 14 provides a partial pivot axis and forms a pivot bearing for the respective pre-centring pin 27. The partial pivot axes (substantially) coincide and provide a common pivot axis.

The glasses frame 1 and the lens arrangement 2 are then pivoted relative to one another in a controlled or guided manner about the common horizontal lower pivot axis predetermined by the pivot bearing regions 14, over a correspondingly predetermined pivot path, with the result that the connecting web 5 and the nose support device 17 approach one another. During the approach, the lens arrangement 2 is located in front of the nose support device 17. Another configuration or design of the lens arrangement 2 and/or nose support device 17 is possible as an alternative.

The nose support device 17 is then pivoted forwards and upwards manually, this being possible by virtue of the twistable frame part 16. The lens arrangement 2 is thus capable of being pivoted past the nose support device 17, below the latter.

After the nose support device 17 has been passed, the nose support device 17 pivots downwards again into its wearing position. This can be done manually. However, the nose support device 17 can also pivot back into its wearing position independently on account of its springback capacity.

The pre-centring means 15 are guided along the inside of the end elements 18 and reach the pre-centring abutment means 30, where they rest, in particular in surface contact.

The latching body 9 latches into the latching recess 26.

The nose support device 17 engages in the nose support device receptacle (FIGS. 1 and 5).

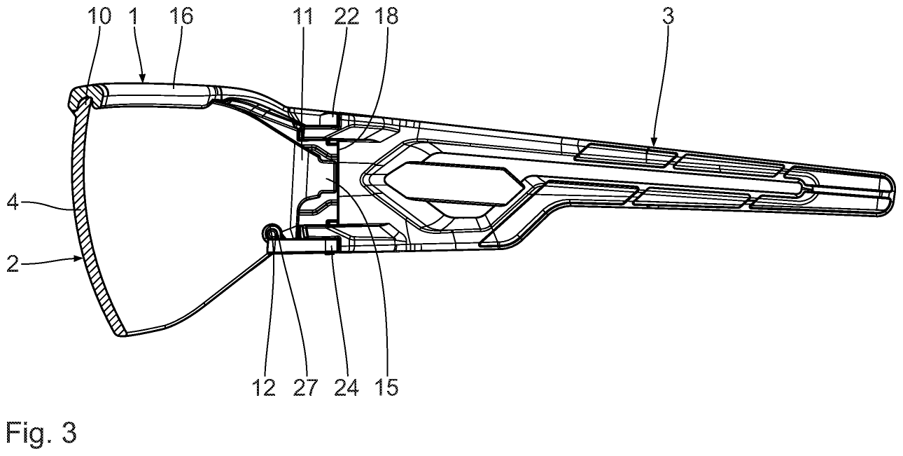

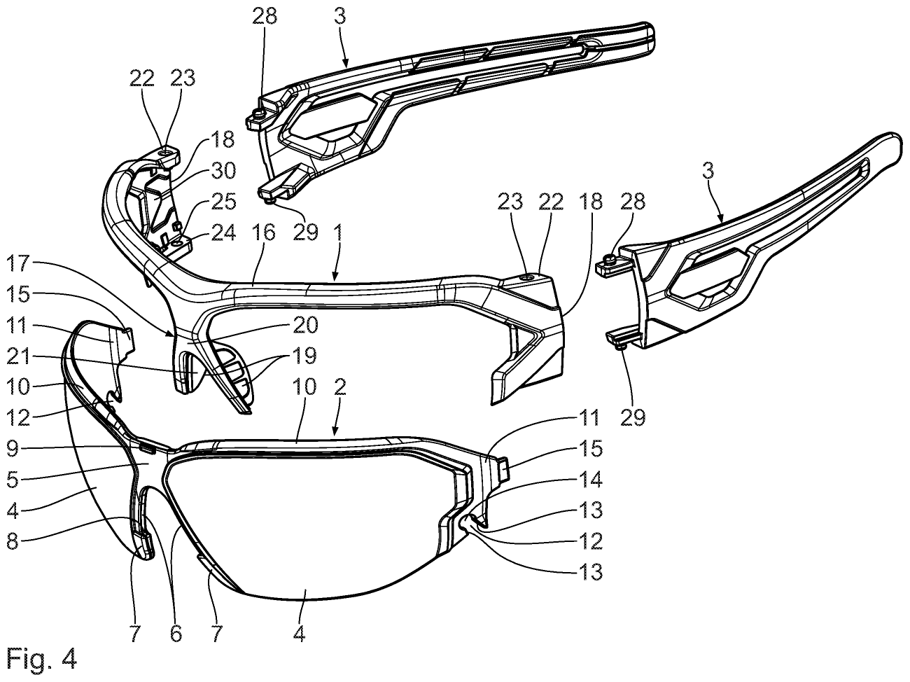

The terminating elements 10 engage in the frame part 16 from below (FIG. 3).

The upper bearing body 28 of each temple 3 is then inserted from below into the upper bearing recess 23, and the lower bearing body 29 is inserted from above into the lower bearing recess 25, thus ensuring that the temple 3 is correspondingly articulated on the glasses frame 1.

In the fully joined-together state, the nose pads 19 adjoin the connecting arms 6 on the inside and preferably also the connecting web 5 at the bottom. Each nose pad 19 is supported on the foot side with respect to the retaining surface 8 of the respective retaining lug 7.

As a result of the engagement of the latching body 9 in the latching recess 26, the lens arrangement 2 is fixed in the upward, downward and lateral directions, and preferably in the forward direction, with respect to the glasses frame 1. As a result of the engagement of the supporting elements 10 from below in the frame part 16, the lens arrangement 2 is fixed in the forward, rearward, upward and, for example, also lateral directions with respect to the glasses frame 1. By virtue of the engagement of the nose support device 17 in the nose support device receptacle, the lens arrangement 2 is fixed in the lateral, downward and preferably upward directions with respect to the glasses frame 1. As a result of the abutment of the pre-centring means 15 against the inside of the end elements 18, the lens arrangement 2 is fixed laterally with respect to the glasses frame 1. The glasses frame 1 and the lens arrangement 2 are braced together in the joined-together state. They are connected detachably to one another.

When the glasses are being worn, the nose pad surfaces 21 rest flat against the nose of the glasses wearer. The nose pads 19 ensure comfortable wearing of the glasses and secure retention. The lenses 4 are located in front of the eyes of the glasses wearer. The temples 3 rest laterally against the head of the glasses wearer.

Disassembly takes place in a substantially analogous manner. In this case, too, the glasses frame 1 and the lens arrangement 2 are pivoted relative to one another in a controlled manner.

What is claimed is:

1. Glasses,
a) having a lens arrangement,
b) having a glasses frame, and
c) having at least one pivot point for joining together the lens arrangement and the glasses frame by pivoting them relative to one another,
d) wherein the glasses frame has a nose support device for support against a nose of a glasses wearer,
e) wherein the lens arrangement has a nose support device receptacle which, in a joined-together state of the lens arrangement and the glasses frame, receives the nose support device.

2. The glasses according to claim 1, wherein the at least one pivot point is predefined.

3. The glasses according to claim 1, wherein the lens arrangement has at least one lower pre-centring pivoting means, and the glasses frame has at least one lower counterpart pre-centring pivoting means for interacting with the at least one lower pre-centring pivoting means upon joining together the lens arrangement and the glasses frame.

4. The glasses according to claim 3, wherein the at least one lower pre-centring pivoting means is designed as a receptacle.

5. The glasses according to claim 4, wherein the at least one lower pre-centring pivoting means is designed as a keyhole-like receptacle.

6. The glasses according to claim 1, wherein the lens arrangement has at least one upper pre-centring means, and the glasses frame has at least one upper counterpart pre-centring means for interacting with the at least one upper pre-centring means upon joining together the lens arrangement and the glasses frame.

7. The glasses according to claim 6, wherein the at least one upper pre-centring means is designed as a protrusion.

8. The glasses according to claim 1, wherein the lens arrangement and the glasses frame are connected to one another by positive engagement in at least one upper glasses region in the joined-together state.

9. The glasses according to claim 1, wherein the nose support device and the nose support device receptacle are connected to one another in a bolt-like manner in the joined-together state of the lens arrangement and the glasses frame.

10. The glasses according to claim 1, wherein the nose support device is pivotable as a whole for joining together of the lens arrangement and the glasses frame.

11. The glasses according to claim 1, wherein the nose support device is pivotable upwards.

12. The glasses according to claim 10, wherein the glasses frame has a frame part which carries the nose support device and is twistable, at least partially, in order to pivot the nose support device.

13. A method for joining together the glasses
a) having a lens arrangement,
b) having a glasses frame, and
c) having at least one pivot point for joining together the lens arrangement and the glasses frame by pivoting them relative to one another,
d) wherein the glasses frame has a nose support device for support against a nose of a glasses wearer,
e) wherein the lens arrangement has a nose support device receptacle which, in a joined-together state of the lens arrangement and the glasses frame, receives the nose support device,
comprising the step of
pivoting the lens arrangement and the glasses frame relative to one another about at least one pivot point.

14. The method according to claim 13, wherein the nose support device is pivoted for the pivoting of the lens arrangement and the glasses frame relative to one another.

15. The method according to claim 13, wherein the nose support device is pivoted upwards, for the pivoting of the lens arrangement and the glasses frame relative to one another.
